SEQUENCE LISTING (10 sequences)

SEQ ID NO: 1 - DNA, 19 bp, synthetic construct
  source 1..19: mol_type = other DNA
gaaggtgaaggtcggagtc

SEQ ID NO: 2 - DNA, 20 bp, synthetic construct
  source 1..20: mol_type = other DNA
gacaagcttcccgttctcag

SEQ ID NO: 3 - DNA, 18 bp, synthetic construct
  source 1..18: mol_type = other DNA
caccagcagcgactctga

SEQ ID NO: 4 - DNA, 22 bp, synthetic construct
  source 1..22: mol_type = other DNA
gatccagactctgaccttttgc

SEQ ID NO: 5 - DNA, 20 bp, synthetic construct
  source 1..20: mol_type = other DNA
ccagggcttttcaaaaatga

SEQ ID NO: 6 - DNA, 20 bp, synthetic construct
  source 1..20: mol_type = other DNA
ccgatccaatctgttctggt

SEQ ID NO: 7 - DNA, 20 bp, synthetic construct
  source 1..20: mol_type = other DNA
ttcacgtgagcacggtaaac

SEQ ID NO: 8 - DNA, 20 bp, synthetic construct
  source 1..20: mol_type = other DNA
gttgggcaacttgtggtctt

SEQ ID NO: 9 - DNA, 21 bp, synthetic construct
  source 1..21: mol_type = other DNA
gagctggtggttgactttctc

SEQ ID NO: 10 - DNA, 21 bp, synthetic construct
  source 1..21: mol_type = other DNA
tccatctccgattcagtccct

(19) United States
(12) Patent Application Publication
(10) Pub. No.: US 2025/0346860 A1
(43) Pub. Date: Nov. 13, 2025

(54) ESTABLISHMENT OF IMMORTALIZED ERYTHROID PROGENITOR CELL LINE WITH SUPERIOR CAPABILITY OF DIFFERENTIATING INTO RED BLOOD CELLS BY USING GENETIC OVEREXPRESSION COMBINATION, PREPARATION METHOD THEREFOR, AND USE THEREOF

(71) Applicants: ARTBLOOD INC., Seoul (KR); IUCF-HYU (Industry-University Cooperation Foundation Hanyang University), Seoul (KR)

(72) Inventors: Eun Jung BAEK, Seoul (KR); Kyeongwon JO, Gyeongsangnam-do (KR); Jumi PARK, Gyeongsangnam-do (KR); Suyeon KIM, Seoul (KR); Hyesook CHOI, Gyeonggi-do (KR); Soonho KWEON, Seoul (KR)

(21) Appl. No.: 18/880,108

(22) PCT Filed: Jul. 4, 2023
(86) PCT No.: PCT/KR2023/009366

§ 371 Date: Feb. 26, 2025

(30) Foreign Application Priority Data
Jul. 4, 2022 (KR) 10-2022-0081790
Jul. 3, 2023 (KR) 10-2023-0086061

(51) Int. Cl. C12N 5/078; C12N 15/86

(52) U.S. Cl. CPC C12N 5/0641; C12N 15/86; C12N 2510/04

(57) ABSTRACT

The present invention relates to the establishment of an immortalized erythroid progenitor cell line and, more specifically, provides an immortalized erythroid progenitor cell line for the production of red blood cells, wherein the progenitor cell line is transducted with c-Myc, BMI1, HOXB4, and BCL-xL.

### TECHNICAL FIELD

The present disclosure relates to the establishment of an immortalized erythroid progenitor cell line, and more specifically, the present disclosure provides an immortalized erythroid progenitor cell line for erythrocyte production transduced with a c-Myc gene, a BMI1 gene, a HOXB4 (Homeobox B4) gene, and a BCL-xL (B-cell lymphoma extra large) gene.

### BACKGROUND ART

Blood transfusion is a very important treatment method for patients who lack blood for various reasons. However, blood donation has problems such as a shortage of blood supply due to a decrease in the number of donors and an aging population. Furthermore, blood donation comes with the risk of spreading infectious diseases through blood transfusion, various other side effects of transfusion, etc. In the case of red blood cell preparations used for the purpose of transporting oxygen among blood components, attempts have been made to use hemoglobin solutions or oxygen carriers as an alternative to the current system of transfusing another person's blood, but the oxygen carrying capacity was low, and serious side effects were shown (Non-patent Documents 1 and 2). Accordingly, research has been continuously conducted on how to produce red blood cells (RBC) in vitro (Non-patent Documents 3 and 4).

In 2005, professor Douay of France published a paper showing the possibility of mass production of red blood cells in vitro from hematopoietic stem and progenitor cells (CD34+ HSCs) (Non-patent Document 5). From that point on, research on in vitro red blood cell production using umbilical cord blood, bone marrow, and peripheral blood have been conducted. However, there is a problem in that mass production is not possible when cells derived from these blood products are cultured in vitro due to limitations on cell proliferation.

In order to solve this problem, research has been conducted to create immortalized cells that overcome the cellular aging process and do not die by overexpressing oncoproteins in erythroid progenitor cells. Representative cell lines include HiDEP (iPSC-derived) and HUDEP (umbilical cord blood CD34+-derived) produced by RIKEN (Rikagaku Kenkyujo) in Japan in 2013, and BEL-A (adult bone marrow CD34+-derived) produced by the University of Bristol in the UK in 2017.

Following the HiDEP, HUDEP, and BEL-A cell lines, which were made with the same vector, follow-up studies to establish human erythroid progenitor cell lines are ongoing worldwide. All the experiments for establishing the three erythroid progenitor cell lines used the CSIV-TRE-HPV16 E6/E7-Ubc-KT vector manufactured by RIKEN to induce HPV16 E6/E7 overexpression.

HUDEP-2 and BEL-A, erythroid progenitor cell lines established in Japan and the UK in 2013 and 2017, respectively, both consist of mixed clonal cell populations. Whereas normal cells have 46 chromosomes (22 pairs of autosomes and 1 pair of sex chromosomes) by two homologous chromosomes being paired, HUDEP-2 has 51 (49-53) modal chromosomes among which partial trisomies (3 chromosomes paired together) were observed. BEL-A has 48 (44-48, XX) chromosomes, among which a high rate of trisomies of chromosomes 6 and 19 were observed (observed in 46 and 45 cells, respectively, out of 50 cells in the metaphase of the cell-division observed).

In a previous study (Non-patent Document 6), the corresponding cell lines could not be maintained by the transduction with c-Myc or BCL-XL alone, due to decreased viability after 14 days. The researchers determined that the decreased mRNA expression of BCL-xL after c-Myc transduction was responsible for the decreased viability of erythroid cell lines. Therefore, they attempted another transfection with a combination of c-Myc and BCL-xL to establish a cell line that proliferated for more than 6 months. However, there was a problem of a low erythrocyte enucleation rate of 0.36%, since induced pluripotent stem cells were used as the cell source, which are known to have a low erythrocyte enucleation rate.

Using HPV 16 E6/E7, a group from the University of Bristol in the UK attempted to immortalize peripheral blood cells, which are CD34 positive, from patients with HbE/β-thalassemia, and the resulting immortalized cell lines showed proliferation of >3 months and an enucleation rate of up to 10% (Non-patent Document 7). Subsequent studies have immortalized cell lines in umbilical cord blood, bone marrow, or CD34+ peripheral blood of healthy donors, and the resulting immortalized cell lines were shown to have a proliferation of >6 months and an enucleation rate of up to 26% (Non-patent Document 8). In addition, a group from Christian Medical University in India attempted to establish cell lines from bone marrow or peripheral blood using HPV16 E6/E7 without using the Tet-On system, and confirmed that proliferation lasted 3 to 4 months and enucleation was efficient at approximately 2 to 21% (Non-patent Document 9). Most recently, a group at the Lund University constructed cell lines from bone marrow cells using E6/E7 without using the Tet-On system, and confirmed that proliferation lasted for more than 11 months, but provided no report on differentiation (Non-patent Document 10).

In order to solve the above mentioned problems, the present inventors added BCL-xL to c-Myc, BMI1 and HOXB4 to produce erythroid progenitor cell lines with normal karyotype and sustained cell proliferation, and created the present disclosure.

### Non-Patent Documents

1. Natanson C et al. JAMA. 2008 May 21; 299 (19): 2304-12.
2. Spahn DR. Crit Care. 1999; 3 (5): R93-7.
3. Migliaccio A R et al. Blood Rev. 2012 March; 26 (2): 81-95.
4. Bouhassira EE. Stem Cells Transl Med. 2012 December; 1 (12): 927-33.
5. Giarratana M C et al. Nat Biotechnol. 2005 January; 23 (1): 69-74.
6. Hirose S et al. Stem Cell Reports. 2013; 1 (6): 499-508
7. Kongtana Trakarnsanga et al. Sci Rep. 2020 Oct. 8; 10 (1): 16798
8. Deborah E Daniels et al. Mol Ther Methods Clin Dev. 2021 Jun. 12; 22:26-39
9. Abhirup Bagchi et al. Cells. 2021 Mar. 1; 10 (3): 523
10. Svetlana Soboleva et al. Hum Cell. 2022 January; 35 (1): 408-417

### DISCLOSURE OF THE INVENTION

### Technical Goals

The technical goal to be achieved by the present disclosure is to provide an immortalized erythroid progenitor cell line.

Another aspect of the present disclosure is providing a method for producing the immortalized erythroid progenitor cell line.

Another aspect of the present disclosure is providing a cell population including erythroid progenitor cells.

Another aspect of the present disclosure is providing a kit for producing the above-mentioned immortalized erythroid progenitor cell line.

Another aspect of the present disclosure is providing a frozen cell composition including the cell population.

Another aspect of the present disclosure is providing erythrocytes differentiated and matured from the above-mentioned immortalized erythroid progenitor cell line; and a cell composition including the erythrocytes.

However, the technical goals to be achieved by the present disclosure are not limited to those mentioned above, and other goals not mentioned will be clearly understood by one of ordinary skill in the art from the following description.

### Technical Solutions

In order to achieve the above mentioned goals, the present disclosure provides an immortalized erythroid progenitor cell line transduced with a c-Myc gene, a BMI1 gene, a HOXB4 (Homeobox B4) gene, and a BCL-XL (B-cell lymphoma extra large) gene.

The BMI1 gene may have 8 folds or less gene expression level than the HOXB4 gene expression level. Preferably, it may be 1 to 8 folds the level or less, more preferably 1.5 to 8 folds the level or less, even more preferably 0.5 to 8 folds the level or less, even more preferably 0.1 to 8 folds the level or less.

The erythrocytes may be derived from mononuclear cells or hematopoietic stem cells.

In another aspect, the present disclosure provides a method for producing an immortalized erythroid progenitor cell line, including the step of transducing a c-Myc gene, a BMI1 gene, a HOXB4 gene and a BCL-xL gene into a monocyte or a hematopoietic progenitor cell in an erythropoietin-containing medium, using a viral vector.

The HOXB4 gene may be introduced at a multiplicity of infection (MOI) two folds higher than the MOI of the c-Myc gene, BMI1 gene, and BCL-xL gene, respectively.

The pre-stimulation may be culturing CD34 positive cells in erythropoietin-containing medium for 1 to 3 days.

The level at which the HOXB4 gene is introduced may be controlled to be at an MOI of 7 to 100 using viruses, and the level at which each of the c-Myc gene, BMI1 gene, and BCL-xL gene is introduced may be controlled to be at an MOI of 3.5 to 50 using viruses.

The erythroid progenitor cell line may be an erythroid cell prior to enucleation.

In another aspect, the present disclosure provides a kit for producing an immortalized erythroid progenitor cell line, including an expression vector expressing a c-Myc gene, a BMI1 gene, a HOXB4 (Homeobox B4) gene, and a BCL-XL (B-cell lymphoma extra large) gene.

The kit may further include an erythroid progenitor cell line proliferating medium including one or more selected from the group consisting of a c-Myc protein or peptide, a BMI1 protein or peptide, a HOXB4 protein or peptide, and a BCL-XL protein or peptide.

In another aspect, the present disclosure provides a cell population including erythroid progenitor cells into which the c-Myc gene, BMI1 gene, HOXB4 (Homeobox B4) gene and BCL-XL (B-cell lymphoma extra large) gene have been introduced.

In another aspect, the present disclosure provides a cell composition including the above-mentioned cell population.

In another aspect, the present disclosure provides erythrocytes differentiated and matured from the immortalized erythroid progenitor cell line according to the present disclosure; and a cell composition including the erythrocytes.

### Effects of the Invention

The immortalized erythroid progenitor cell lines derived from CD34 positive cells, transduced with the c-Myc, BMI1, HOXB4 and BCL-xL genes of the present disclosure provide sustained cell proliferation effects.

The immortalized erythroid progenitor cell lines according to the present disclosure provide the effect of maintaining ≥90% of cell viability and continuously proliferating them even when the cell lines are thawed and cultured after cryopreservation.

The immortalized erythroid progenitor cell lines according to the present disclosure may be utilized to produce erythrocytes for transfusion in humans as well as in pets such as dogs, cats, and guinea pigs, or for reagent use (such as for detecting erythrocyte antibodies).

### BRIEF DESCRIPTION OF THE DRAWINGS

FIGS. 1A and 1B show results that show the effect of overexpression of four genes (c-Myc, BMI1, HOXB4, and BCL-xL) on cell proliferation.

FIGS. 2A and 2B show the differentiation of erythroid cells, confirmed by Wright-Giemsa Stain.

FIGS. 3A and 3B are graphs that show the differentiation rate of erythroid cells, obtained by Wright-Giemsa Stain.

FIGS. 4 and 5 show the results of comparing the expression levels of BMI1 and HOXB4 in erythroid progenitor cell lines.

FIGS. 6A and 6B show the results of experiments performed repeatedly in which overexpression of four genes (c-Myc, BMI1, HOXB4, and BCL-xL) resulted in proliferation and the results of comparing the levels of expression of BMI1 and HOXB4 in erythroid progenitor cell lines.

FIG. 7 shows the effect of overexpression of four genes (c-Myc, BMI1, HOXB4, and BCL-xL) on cell proliferation by expanding the cell source to include one derived from bone marrow.

FIG. 8 shows the effect of overexpression of four genes (c-Myc, BMI1, HOXB4, and BCL-xL) on cell survival rate by expanding the cell source to include one derived from bone marrow.

FIGS. 9A and 9B are graphs showing the proliferation rate and survival rate of cells selected by culturing them as single cells after overexpression of four genes (c-Myc, BMI1, HOXB4, and BCL-xL) for 48 days.

### BEST MODE FOR CARRYING OUT THE INVENTION

The present disclosure provides an immortalized erythroid progenitor cell line transduced with a c-Myc gene, a BMI1 gene, a HOXB4 (Homeobox B4) gene, and a BCL-xL (B-cell lymphoma extra large) gene.

The BMI1 gene may have 0.5 to 8 folds or less gene expression level than the HOXB4 gene expression level.

The erythroid cell may be derived from mononuclear cells or hematopoietic stem cells.

As used herein, the term “hematopoietic stem cells (HSCs)” refers to cells that may potentially differentiate into myeloid or erythroid cells found in blood, such as erythrocytes, T cells, neutrophils, granulocytes, monocytes, natural killer cells, basophils, dendritic cells, eosinophils, mast cells, B cells, platelets, and megakaryocytes. For example, the hematopoietic stem cells may be isolated from human-derived umbilical cord blood, peripheral blood, or bone marrow-derived cells, and preferably, may be isolated from human umbilical cord blood derived cells.

As used herein, the term “erythroid progenitor cell” means an erythroid cell before enucleation, which may be an immature erythroid progenitor cell or a pre-enucleation cell that is negative for Glycophorin A (GPA), a molecule specific for the erythroid lineage, wherein CD71 is positive, and GPA may be partially positive or negative depending on the culture period.

Erythrocytes (RBC) are produced from hematopoietic stem cells through several stages of differentiation: burst forming unit erythroid (BFU-E), colony forming unit erythroid (CFU-E), proerythroblast, basophilic erythroblast, polychromatic erythroblast, and orthochromatic erythroblast, during which the nucleus gradually condenses and the cell size decreases. After the condensed nucleus is enucleated, it becomes a reticulocyte. When the remaining RNA and micro-organelles disappear and both sides become concave, it becomes a mature RBC.

As used herein, the term “cell” means a cell derived from human and non-human animals (e.g., mice, rats, cows, horses, pigs, sheep, monkeys, dogs, cats, birds, etc.), and is not particularly limited, but preferably means a cell derived from a human.

According to an embodiment of the present disclosure, the erythroid progenitor cell lines of the present disclosure is derived from CD34 positive cells derived from human umbilical cord blood hematopoietic stem cells.

c-Myc is widely known as a factor that regulates apoptosis, proliferation, and differentiation in hematopoietic stem cells, and there is an example of an erythroid progenitor cell line that was established by inducing overexpression of c-Myc and Bcl-XL in induced pluripotent stem cells. However, this cell line has a slow doubling time of approximately 40 hours and a very low enucleation rate in vitro. In addition, it is known that c-Myc causes apoptosis, and thus, in hematopoietic stem cells, c-Myc is not sufficient to create cell lines from hematopoietic stem cells, so other additional genes are required.

BMI1 and HOXB4 (Homeobox B4) are known to maintain the self-renewal of hematopoietic stem cells by inhibiting the expression of p16 and p19, and the expression of p21 and p27, respectively. HOXB4 overexpression is known to allow hematopoietic progenitor cells to proliferate by downregulating Gemini proteins. (HoxB4 transduction down-regulates Geminin protein, providing hematopoietic stem and progenitor cells with proliferation potential, PNAS, 2010, Vol. 107, No. 50 (Dec. 14, 2010), pp. 21529-21534). However, in the present disclosure, when HOXB4 and BMI1 were overexpressed simultaneously, they showed a pattern of inhibiting each other's expression levels. This is because even at higher MOls, the amount of each vector entering the cell is limited, and because some of the intracellular downstreams that are utilized for proliferation by both genes may be partially shared. Nonetheless, proliferation is maximized when HOXB4 and BMI1 are present in an appropriate ratio, which appears to be more advantageous for maintaining cell line and differentiation potency than any other combination.

BCL-xL (B-cell lymphoma extra large) is known to inhibit apoptosis by preventing the efflux of mitochondrial components that induce apoptosis, such as cytochrome c.

The erythroid progenitor cell line of the present disclosure may be produced by transducing a c-Myc gene, a BMI1 gene, a HOXB4 gene, and a BCL-XL gene into CD34 positive cells that are pre-stimulated in an erythropoietin (EPO)-containing medium.

The pre-stimulation involves culturing CD34 positive cells in erythropoietin-containing medium for 1 to 3 days, which allows CD34 positive cells to change their lineage to erythroid cell lineage (i.e., erythroid cells after differentiation).

The transduction is performed by transfecting pre-stimulated CD34 positive cells with a viral vector containing the c-Myc gene, BMI1 gene, HOXB4 gene, and BCL-xL gene. The efficiency of transducing the genes into CD34 positive cells may be improved by controlling the MOI of the viral vector and the number of transductions.

Additionally, the HOXB4 gene may be introduced at a multiplicity of infection (MOI) two folds higher than the MOI of the c-Myc gene, BMI1 gene, and BCL-xL gene, respectively.

According to an embodiment of the present disclosure, the level at which the HOXB4 gene is introduced may be controlled to be at an MOI of 7 to 100 using viruses, and the levels at which each of the c-Myc gene, BMI1 gene, and BCL-XL gene is introduced may be controlled to be at an MOI of 3.5 to 50 using viruses.

According to an embodiment of the present disclosure, human umbilical cord blood CD34 positive cells are pre-stimulated with an erythropoietin-containing medium for 24-72 hours, then the cells are transduced with lentiviral particles including a target gene for 24 hours, which is followed by antibiotic screening to obtain an erythroid progenitor cell line.

As used herein, the term “MOI” means “multiplicity of infection.” For example, if 1,000,000 vectors were used to transduce 100,000 host cells, the MOI is 10. When using a viral vector, MOI represents the ratio of the amount of inoculated virus to the number of cells inoculated with the virus, i.e. the average number of virus particles that infect a single cell. The cases in which this term may be used are not limited to cases involving transduction, but also include cases where vectors are introduced into a host cell by means such as lipofection, microinjection, calcium phosphate precipitation, and electroporation.

As used herein, the term “vector” refers to a nucleic acid molecule capable of transporting another nucleic acid linked thereto. One type of vector is a plasmid, which is a circular double-stranded DNA loop into which an additional DNA segment may be ligated. Another type of vector is a viral vector, which allows an additional DNA segment to be ligated into a viral genome. Some vectors are capable of self-replication within a host cell when transduced into the host cell (e.g., bacterial vectors having a bacterial origin of replication and episomal mammalian vectors). Other vectors (e.g., non-episomal mammalian vectors) may be integrated into a host cell's genome when transduced into the host cell and replicate along with the host genome. In addition, some vectors may direct the expression of genes to which the vectors are operably linked. In the present specification, these vectors are referred to as recombinant expression vectors (or simply, expression vectors). In general, expression vectors useful in recombinant DNA techniques are usually in the form of plasmids, and since plasmids are the most commonly used vector type, the terms “plasmid” and “vector” may be used interchangeably. However, the present disclosure also includes other forms of expression vectors such as viral vectors (e.g., adenovirus vectors, adeno-associated virus (AAV) vectors, herpes virus vectors, retroviral vectors, lentiviral vectors, baculovirus vectors) that provide equivalent functions. Preferably, a retroviral vector may be used. Preferably, a lentiviral vector may be used to increase the transduction efficiency of CD34 positive cells.

In addition, it is preferable that the vector further include a selection marker. As used herein, the term “selection marker” refers to a marker used to facilitate selection of transformed cells into which the c-Myc gene, BMI1 gene, and HOXB4 gene expression cassettes are transduced. A selection marker that may be used in the vector of the present disclosure is not particularly limited as long as it is a gene that allows easy detection or measurement of whether the vector has been transduced, but typical examples include markers that impart selectable phenotypes such as drug resistance, auxotrophy, resistance to cytotoxic agents, or surface protein expression, for example, blasticidin, doxycycline, GFP (green fluorescent protein), puromycin, neomycin (Neo), hygromycin (Hyg), histidinol dehydrogenase gene (hisD), or guanine phosphoribosyltransferase (Gpt).

The gene transduction includes any method of transducing a nucleic acid into an organism, cell, tissue or organ, and may be performed by selecting an appropriate standard technique depending on the host cell, as known in the art. These methods include electroporation, protoplast fusion, calcium phosphate (CaPO₄) precipitation, calcium chloride (CaCl₂)) precipitation, agitation using silicon carbide fibers, agrobacteria-mediated transformation, polyethylene glycol (PEG), dextran sulfate, and Lipofectamine, but are not limited thereto.

The c-Myc gene, BMI1 gene, HOXB4 gene, and BCL-XL gene used in the present disclosure include not only genes whose cDNA sequences have already been disclosed, but also homologs identified by the related art based on the homology of these known cDNA sequences.

CD34 positive cells transduced with the c-Myc gene, BMI1 gene, HOXB4 gene, and BCL-XL gene of the present disclosure were cultured in a medium for maintaining a cell line (StemSpan SFEM (Stemcell Technologies)) containing 3 IU/ml EPO, 50 ng/ml SCF, 1 μM Dexamethasone (Sigma), and 1 μg/mL Doxycycline (Takara Bio). About 2 to 3 days after gene introduction, the erythroid progenitor cells with a high proliferative capacity were acquired. The pre-enucleated erythroid progenitor cells obtained in this manner continued to proliferate for at least 30 to 50 days, preferably for at least 50 to 60 days, more preferably for 60 days or more, and the number of cells increased about 1.0×10³ times or more, preferably about 1.0×10⁴ times or more, more preferably about 1.0×10⁶ times or more, than the number of cells at the time the c-Myc gene, BMI1 gene, HOXB4 gene, and BCL-xL gene were introduced.

In addition, the present disclosure provides a kit for producing an immortalized erythroid progenitor cell line, including an expression vector expressing a c-Myc gene, a BMI1 gene, a HOXB4 gene, and a BCL-xL gene.

The kit includes, in addition to expression vectors and reagents necessary for expressing the c-Myc gene, BMI1 gene, HOXB4 gene, and BCL-xL gene in cells, cell culture media serum, supplements such as growth factors (e.g., EPO, SCF, Dexamethasone, etc.), antibiotics, etc. Other examples include antibodies for identifying markers for immortalized erythroid progenitor cell lines. The reagents, antibodies, and the like included in the kit are supplied in any container in which the components remain active for a long period of time, are not adsorbed by the material of the container, and do not deteriorate.

The cell culture medium, which is a proliferation medium, may further include an erythroid progenitor cell line proliferating medium including one or more selected from the group consisting of a c-Myc protein or peptide, a BMI1 protein or peptide, a HOXB4 protein or peptide, and a BCL-xL protein or peptide.

The erythroid progenitor cells of the present disclosure may be differentiated into mature erythroid cells by culturing them in a medium for differentiation and maturation. The medium for differentiation and maturation may be a cell culture medium to which any one of TPO, IL-1α, IL-3, IL-4, IL-5, IL-6, IL-9, IL-11, EPO, GM-CSF, SCF, G-CSF, Flt3 ligand, heparin, and the like, or a combination of two or more of these is added. In particular, RBCs may be cultured for about 7 to 15 days in the presence of EPO (2 to 100 U/mL, preferably about 10 U/mL) or in the presence of EPO (2 to 100 U/mL, preferably about 10 U/mL) and SCF (10 to 200 ng/ml, preferably about 50 ng/ml). The culture environment may be any suitable environment for inducing differentiation of red blood cells in vitro. For example, the culture may be performed under conditions of 5% CO₂ and 36 to 38° C., preferably 37° C.

In addition, the present disclosure provides a cell population including erythroid progenitor cells into which the c-Myc gene, BMI1 gene, HOXB4 gene and BCL-xL gene have been introduced.

Furthermore, the present disclosure provides a cell composition including the cell population of the immortalized erythroid progenitor cell line.

In addition, the present disclosure provides erythrocytes differentiated and matured from the above-mentioned immortalized erythroid progenitor cell line according to the present disclosure; and a cell composition including the erythrocytes.

The cell composition may include the immortalized erythroid progenitor cell lines or erythrocytes, either in a cryopreserved state or at room temperature or in a refrigerated state.

Furthermore, the immortalized erythroid progenitor cell lines of the present disclosure are freeze-thaw resistant, retaining their proliferation and differentiation capacities upon thawing after cryopreservation. As such, it is possible to freeze the erythroid progenitor cell line and thaw it as needed to produce differentiated hematopoietic cells. Thus, by utilizing the present cells, there is no need to perform a series of steps to produce hematopoietic cells, such as platelets, from CD34 positive cells from the beginning. In other words, by preparing a large amount of erythroid progenitor cell lines into which the c-Myc gene, BMI1 gene, HOXB4 gene, and BCL-xL gene of the present disclosure have been introduced as raw materials, and cryopreserving them as needed, the manufacturing process may be streamlined and made more efficient, and thereby a system for rapidly supplying various hematopoietic cells, such as platelets, may be established.

When the immortalized erythroid progenitor cell line or erythrocytes of the present disclosure are used to make the frozen cell composition, the composition may contain, in addition to the erythroid progenitor cell line, any media or buffers necessary for preserving the cells, and other substances necessary to protect the cells during freezing, such as DMSO, glycerol, etc. In addition, any other substances normally required for cell freezing may be included. Alternatively, if commercially available reagents for freezing cells are used, the composition may contain any substances contained in the reagents.

Hereinafter, embodiments will be described in detail with reference to the accompanying drawings. However, since various changes may be made to the embodiments, it will be understood that the descriptions of the embodiments do not limit or otherwise restrict the scope of the patent application. With respect to the embodiments, it should be understood that the scope of the rights shall include all modifications, equivalents and substitutes. With respect to the description of an embodiment, in case where it was determined that including a detailed description of a related known technology may unnecessarily obscure the gist of the embodiment, such detailed description was omitted.

### EXAMPLES

Hereinafter, the present disclosure will be described in detail by examples.

However, the following examples only illustrate the present disclosure, and the present disclosure is not limited to the following examples.

### <Example 1> Transduction for Establishment of Erythroid Progenitor Cell Lines

1-1: Isolation of Hematopoietic Stem Cells (CD34 Positive Cells) Derived from Umbilical Cord Blood

The umbilical cord blood used in the present disclosure was received from a healthy mother who gave written consent to donate in advance. Umbilical cord blood mononuclear cells were isolated through centrifugation using Ficoll-Hypaque. Then, CD34 positive cells were isolated from the mononuclear cells using a CD34 MicroBead Kit (Miltenyi Biotec, Germany) and MS Column (Miltenyi Biotec). The present disclosure was created after approval from the Institutional Review Board of Hanyang University was received.

### 1-2: Study of Conditions for Cell Line Establishment

### 1-2-1: Lentivirus Particle Production and Titer Measurement

293 FT cells were cultured in Dulbecco's modified Eagle's medium (DMEM; Gibco, Grand Island, NY) supplemented with 10% fetal calf serum (Gibco) (not inactivated by heat treatment), 0.1 mM MEM Non-Essential amino acids (NEAA) (Gibco), 6 mM L-glutamine (Gibco), and 500 μg/ml Geneticin (Gibco). Cryopreserved 293 FT cells were thawed and used for transfection after three passages. One day before transfection, Geneticin was removed from the culture medium and the cells were seeded in a T75 flask at 6×10 cells/12 mL. Using Lipofectamine™ 2000 transfection reagent (Invitrogen, Carlsbad, CA), vectors containing eGFP (enhanced green fluorescent protein), eGFP-c-Myc, eGFP-BMI1, eGFP-HOXB4, and BCL-xL were transduced into 293 FT cells together with the lentiviral packaging plasmids psPAX2 and pMD2.G (Addgene). The medium was replaced with a fresh medium after 12 hours.

The sequence information used for the expression of the target genes c-Myc, BMI1, HOXB4 and BCL-xL is as follows:
c-Myc: NCBI accession no. NM_002467.6
BMI1: NCBI accession no. NM_001204062.1
HOXB4: NCBI accession no. NM_024015.4
BCL-xL: NCBI accession no. NM_138578.3
24 hours after the medium change, the supernatant containing viral particles was collected, centrifuged (3,000 rpm, 15 min, 4° C.), and then filtered through a 0.45 μm filter. The supernatant containing the produced viral particles was concentrated by ultracentrifugation (20,000 g, 4 hours, 4° C.). Afterwards, 5×10⁴ cells/0.5 mL cultured from CD34 positive cells in 48 well plates were transduced with medium supplemented with polybrene (8 μg/mL) using a 5-fold serial dilution method. The next day, the medium was changed to a fresh medium supplemented with 1 μg/mL Doxycycline. On day 3 of culture, the level of GFP fluorescence expression in the cells was measured by flow cytometry. Then, the titer was calculated from the measured value of GFP.
   1-2-2: Culture after Transduction

CD34 positive cells isolated from umbilical cord blood (day 0) were cultured in pre-stimulation medium for 3 days, and transduction was performed with viruses carrying c-Myc, BMI1, HOXB4, and BCL-xL genes together with viruses carrying rtTA (reverse tetracycline-controlled transcriptional activator) using medium supplemented with polybrene (8 μg/mL). The pre-stimulation medium was composed of Iscove's Modified Dulbecco's Medium (IMDM) supplemented with 5% fetal bovine serum, 10 μg/mL insulin, 3 U/mL EPO, 200 μg/mL transferrin, 10 ng/mL SCF, and 1 ng/ml IL-3.

Cells were washed 24 hours after transduction. 48 hours after transduction, the medium was replaced with growth medium (Stemspan, EPO, SCF, Dexamethasone) containing 1 μg/mL Doxycycline and the cells were cultured. Afterwards, the medium was exchanged every 2-3 days, and the cell counts, viability tests, and the GFP fluorescence expression rates were checked at each change.

1-2-3: qRT-PCR

Quantitative real time RT-PCR (qRT-PCR) was performed to confirm the expression level of mRNA of the c-Myc, BMI1, HOXB4, and BCL-xL genes transduced for cell line production.

RNA was isolated from the harvested cells using Trizol Reagent (Ambion, Austin, USA), and the RNA concentration was measured using a SpectraMax® ABS Plus absorbance microplate reader (Molecular Devices). cDNA was synthesized using the SuperScript III First-Strand Synthesis System (Invitrogen), and mRNA expression was measured in duplicate by qRT-PCR using SYBR green (TOPreal qPCR 2× PreMIX; Enzynomics, Daejeon, Korea). The values were corrected using GAPDH, and the primer sequences used in the experiment are as follows (forward/reverse):

GAPDH,
(SEQ ID NO: 1)
5′-GAAGGTGAAGGTCGGAGTC-3′/
(SEQ ID NO: 2)
5′-GACAAGCTTCCCGTTCTCAG-3′
c-Myc,
(SEQ ID NO: 3)
5′-CACCAGCAGCGACTCTGA-3′/
(SEQ ID NO: 4)
5′-GATCCAGACTCTGACCTTTTGC-3′
BMI1,
(SEQ ID NO: 5)
5′-CCAGGGCTTTTCAAAAATGA-3′/
(SEQ ID NO: 6)
5′-CCGATCCAATCTGTTCTGGT-3′
HOXB4,
(SEQ ID NO: 7)
5′-TTCACGTGAGCACGGTAAAC-3′/
(SEQ ID NO: 8)
5′-GTTGGGCAACTTGTGGTCTT-3′
BCL-xL,
(SEQ ID NO: 9)
5′-GAGCTGGTGGTTGACTTTCTC-3′/
(SEQ ID NO: 10)
5′-TCCATCTCCGATTCAGTCCCT-3′

### <Example 2> Confirmation of the Effect of Overexpression of Four Genes (c-Myc, BMI1, HOXB4, and BCL-xL) on Cell Proliferation

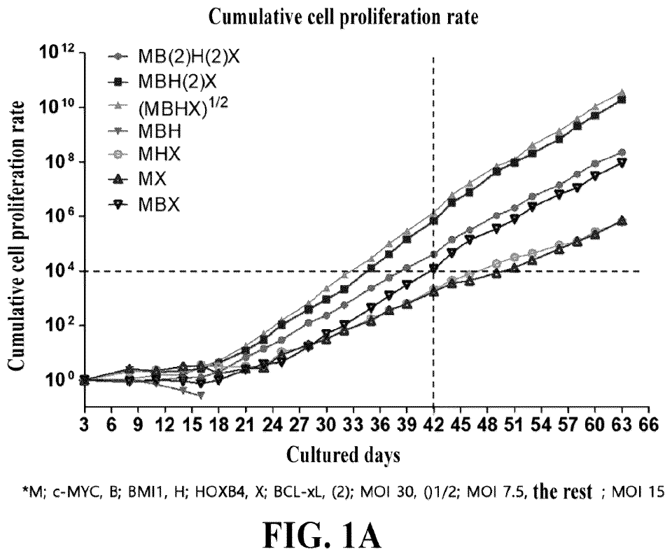

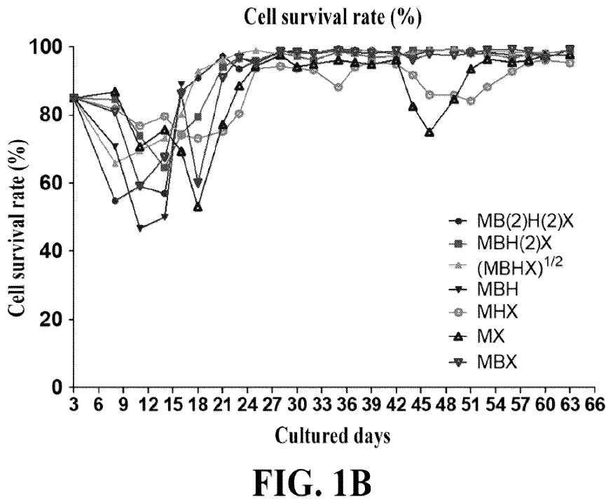

The proliferation rates of cell lines overexpressed with all the four genes c-Myc, BMI1, HOXB4, and BCL-xL were compared to those under the conditions of existing Patent Document 1 (KR10-2273668) for establishing an erythroid progenitor cell line by overexpressing c-Myc, BMI1, and HOXB4; existing Patent Document 2 (U.S. Pat. No. 10,087,419) for establishing an erythroid progenitor cell line by overexpressing c-Myc and BCL-xL; and existing Patent Document 3 (U.S. Pat. No. 9,200,254) for establishing a platelet progenitor cell line by overexpressing c-Myc, BMI1, and BCL-xL. M represents c-Myc, B represents BMI1, H represents HOXB4, and X represents BCL-xL. “(2)” represents treatment with viruses at an MOI of 30, and “( )^{1/2}” represents treatment with viruses at an MOI of 7.5, and the rest represent treatment with viruses at an MOI of 15. As a result, the MBH conditions induced cell death on day 17 of culture and the culture was terminated, and the remaining conditions showed stable proliferation with a survival rate of more than 90% for more than 60 days (FIGS. 1A and 1B). Among them, when cultured for up to 60 days, the conditions resulting in proliferation of more than 10⁶ times were MB(2)H(2)X, MBH(2)X, (MBHX)^{1/2} conditions (all four genes were treated to be overexpressed), and MBX conditions (FIG. 1A). In order to select the erythroid progenitor cell line with the highest differentiation rate into erythrocytes, differentiation experiments were conducted.

### <Example 3> Selecting the Optimal Erythroid Progenitor Cell Line Through Confirmation on Differentiation into Erythrocytes

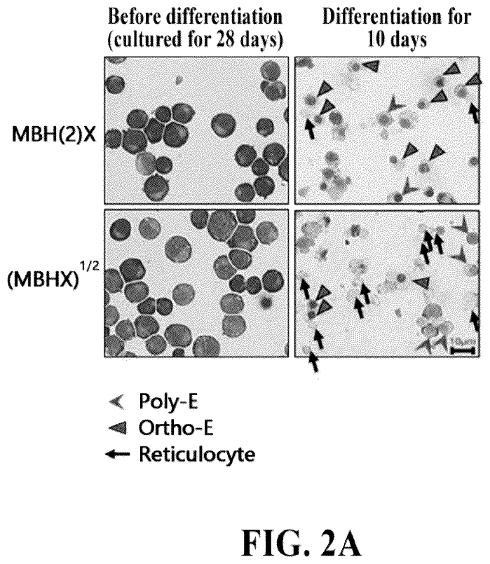

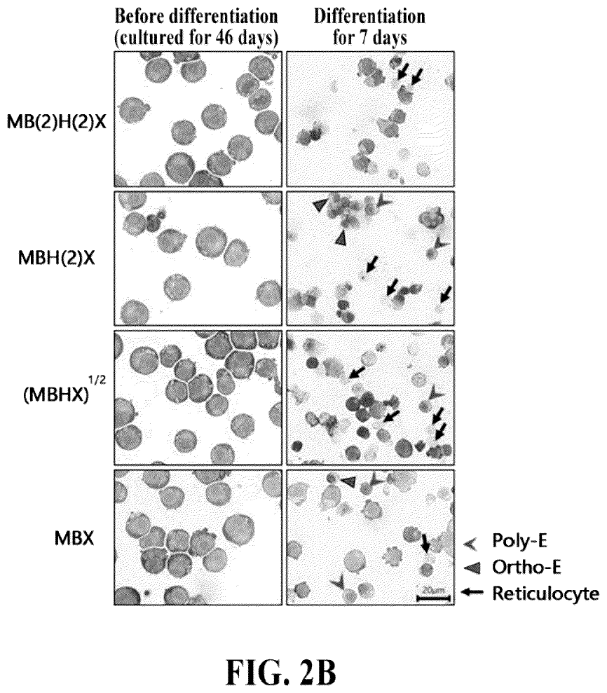

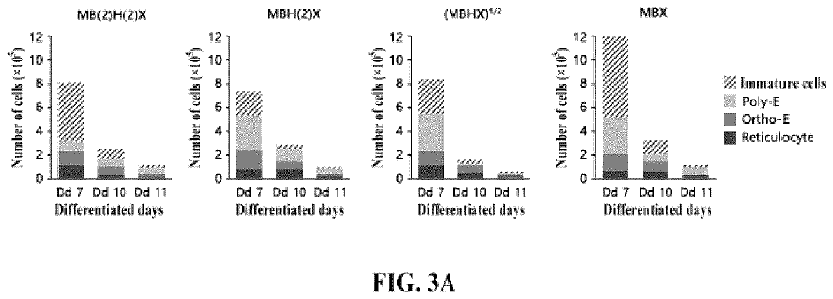

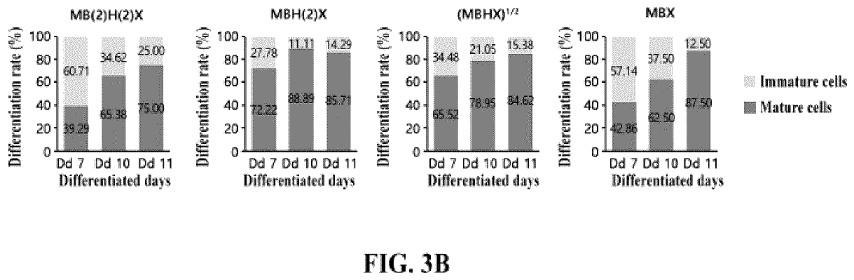

For the four conditions that showed proliferation of 10⁴ times or more as of 42 days of culture, i.e., MB(2)H(2)X, MBH(2)X, (MBHX)^{1/2} and MBX, the medium was changed to a differentiation medium on day 46 of culture, and Doxycycline was removed on day 49 of culture (differentiation day 3). The differentiation rate of erythroid cells was analyzed through Wright-Giemsa staining on days 7, 10, and 11 of differentiation. The composition of the differentiation medium included IMDM medium supplemented with 10% fetal bovine serum, 10 μg/mL insulin, and 3 U/mL EPO. 10 ng/ml SCF and 5 ng/ml IL-3 were added only until differentiation day 4; 330 μg/mL transferrin was added until differentiation day 9; and 500 μg/mL transferrin was added thereafter. FIG. 2A shows the Wright-Giemsa staining images of MBH(2)X and (MBHX)^{1/2} conditions, differentiated at the beginning of culture, day 10. Mature erythroid cells were divided into Poly-E (polychromatic erythroblast), Ortho-E (orthochromatic erythroblast), and Reticulocyte. FIG. 2A shows representative images thereof. FIG. 2B shows the Wright-Giemsa staining images taken at day 7 of differentiation after differentiation on day 46 of culture. FIG. 3A is a graph showing the number of erythroid cells at each differentiation stage differentiated on day 46 of culture; and FIG. 3B is a graph that shows the differentiation rate as a plot of the ratio of immature cells and mature cells (Poly-E, Ortho-E, and Reticulocyte). The analysis shows that MBH(2)X has a differentiation rate of more than 80% from day 10 of differentiation, while (MBHX)^{1/2} has a differentiation rate of more than 80% on day 11 of differentiation. Additionally, the analysis shows that the cell survival rate of MBH(2)X during differentiation was about 2 folds higher than that of (MBHX)^{1/2}.

### <Example 4> Comparison of Expression Levels of BMI1 and HOXB4 in Erythroid Progenitor Cell Lines

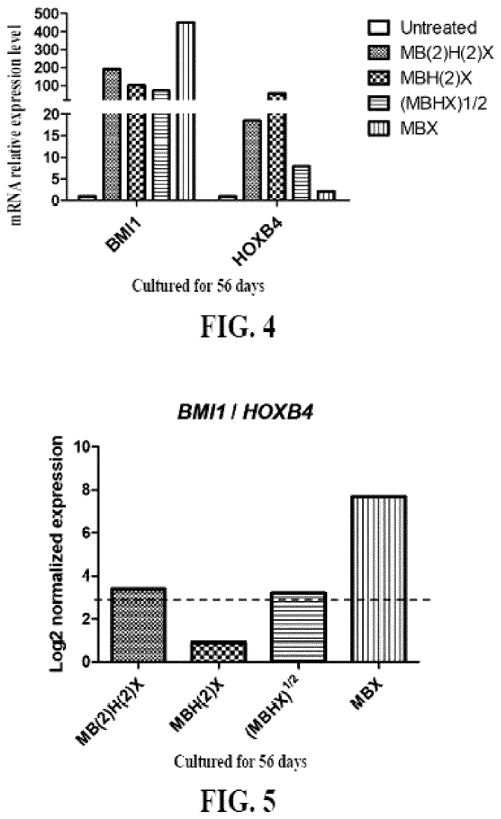

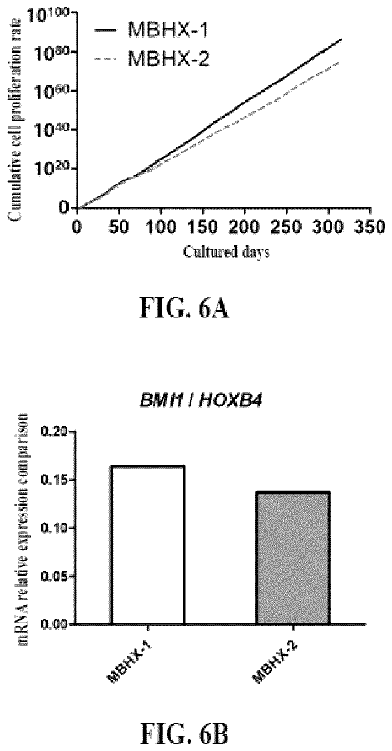

To determine what differences among the established erythroid progenitor cell lines led to differences in erythroid differentiation rates, RNA was extracted from cells cultured for 56 days and the mRNA expression levels of the transduced genes were compared to each other (FIG. 4). As a result, when the expression level of BMI1 was compared to that of HOXB4, a difference was observed under the MBH(2)X condition. Specifically, in the case of MBH(2)X, BMI1 was 1.8 folds (log 2 fold change=0.9) higher than HOXB4; and in the case of MB(2)H(2)X, (MBHX)^{1/2}, and MBX, BMI1 was 10.4 folds (log 2 fold change=3.4), 8.9 folds (log 2 fold change=3.2), and 210 folds (log 2 fold change=7.7) higher than HOXB4, respectively (FIG. 5). As such, it can be seen that a high level of BMI1 expression may have a positive effect on cell proliferation but a negative effect on differentiation into erythrocytes. Thus, in order to establish an erythroid progenitor cell line with a high erythroid differentiation rate and a high cell proliferation rate, it is important to maintain the expression level of BMI1 at less than 8 folds that of HOXB4. FIG. 6A shows a graph of cell proliferation rate in replicate experiments using overexpression of four genes (c-Myc, BMI1, HOXB4, and BCL-XL); and FIG. 6B shows a graph comparing the mRNA expression of HOXB4 and BMI1, showing that the expression level of BMI1 is less than 0.17 folds that of HOXB4.

### <Example 5> Establishment of Erythroid Progenitor Cell Line Using Bone Marrow Derived CD34 Positive Cells

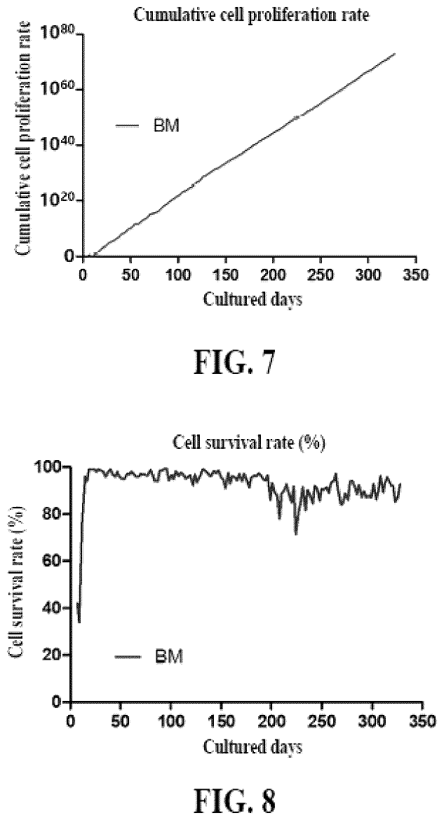

Previously, umbilical cord blood-derived CD34+ cells were cultured for 3 days, which was followed by the overexpression of four genes c-Myc, BMI1, HOXB4, and BCL-xL in the cells to perform a cell line establishment study. In order to perform the cell line establishment study in the same manner but with a different cell source, bone marrow-derived (BM) hematopoietic stem cell CD34 positive cells were cultured for 3 days and then overexpressed the four genes therein. The bone marrow-derived erythroid progenitor cell lines proliferated continuously with a survival rate of more than 85%, increasing 1078-fold on day 328 of culture (FIGS. 7 and 8). The results demonstrated that erythroid progenitor cell lines may be established by transducing four genes (c-Myc, BMI1, HOXB4, and BCL-XL) into the corresponding cell source using various cell sources.

### <Example 6> Single Cell Culture from Erythroid Progenitor Cell Lines

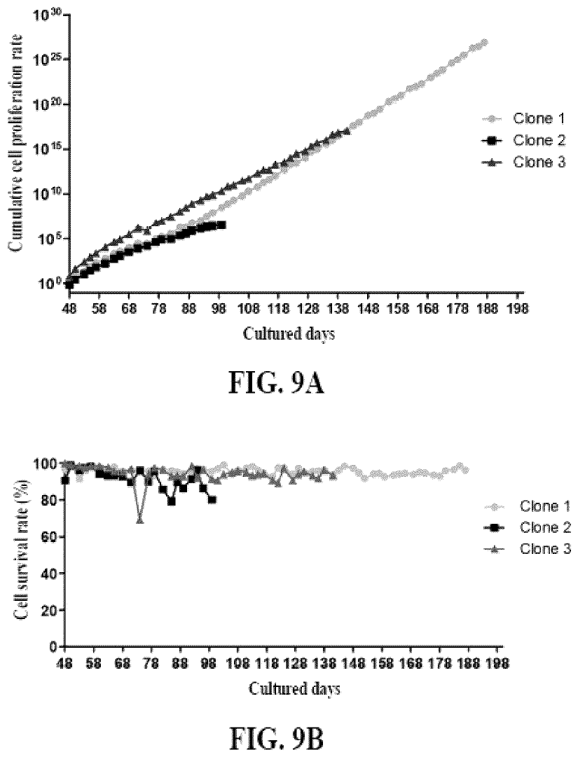

FIGS. 9A and 9B are graphs representing the cell proliferation and survival rates when umbilical cord blood derived CD34 positive cells overexpressing the four genes were cultured for up to 48 days, and then were isolated and cultured as single cells. The results show that the established erythroid progenitor cell lines may be cultured as single cells.

While certain aspects of the present disclosure have described in detail, it will be apparent to one of ordinary skill in the art that they are merely illustrative, and one of ordinary skill in the art will understand that these specific descriptions are merely descriptions of preferred embodiments and are not intended to limit the scope of the present disclosure. Accordingly, the scope of the present disclosure should substantially be defined by the appended claims and their equivalents.

This disclosure was made with the support of the government ministry, Ministry of SMEs and Startups (Project No. S3307527/Title: Development of cell-based in vitro artificial red blood cells).

1. An immortalized erythroid progenitor cell line transduced with a c-Myc gene, a BMI1 gene, a HOXB4 (Homeobox B4) gene, and a BCL-XL (B-cell lymphoma extra large) gene.

2. The immortalized erythroid progenitor cell line of claim 1, wherein the BMI1 gene has 8 folds or less gene expression level than the HOXB4 gene expression level.

3. The immortalized erythroid progenitor cell line of claim 1, wherein the erythroid progenitor cells are derived from mononuclear cells or hematopoietic stem cells.

4. A method for producing an immortalized erythroid progenitor cell line, comprising:
transducing a c-Myc gene, a BMI1 gene, a HOXB4 gene and a BCL-xL gene into a monocyte or a hematopoietic progenitor cell in an erythropoietin-containing medium, using a viral vector,
wherein the HOXB4 gene is introduced at a multiplicity of infection (MOI) two folds higher than the MOI of the c-Myc gene, BMI1 gene, and BCL-xL gene, respectively.

5. The method of claim 4, wherein the level at which the HOXB4 gene is introduced is controlled to be at an MOI of 7 to 100 using viruses, and the level at which each of the c-Myc gene, BMI1 gene, and BCL-XL gene is introduced is controlled to be at an MOI of 3.5 to 50 using viruses.

6. The method of claim 4, wherein the immortalized erythroid progenitor cell line is an erythroid cell line prior to enucleation.

7. A kit for producing an immortalized erythroid progenitor cell line, comprising an expression vector expressing a c-Myc gene, a BMI1 gene, a HOXB4 (Homeobox B4) gene, and a BCL-XL (B-cell lymphoma extra large) gene.

8. The kit of claim 7, wherein the kit further comprises an erythroid progenitor cell line proliferating medium comprising one or more selected from a group consisting of a c-Myc protein or peptide, a BMI1 protein or peptide, a HOXB4 protein or peptide, and a BCL-xL protein or peptide.

9. A cell population comprising erythroid progenitor cells into which a c-Myc gene, a BMI1 gene, a HOXB4 (Homeobox B4) gene and a BCL-XL (B-cell lymphoma extra large) gene have been introduced.

10. A cell composition comprising the cell population of claim 9.

11. An erythrocyte differentiated and matured from the immortalized erythroid progenitor cell line of claim 1.

12. A cell composition comprising the erythrocyte of claim 11.
